(19) United States
(12) Patent Application Publication
(10) Pub. No.: US 2017/0354528 A1
(43) Pub. Date: Dec. 14, 2017

(54) MOLDABLE WRIST BRACE

(71) Applicant: Scott Specialties, Inc., Belleville, KS (US)

(72) Inventors: Meghan Lane, Cavan (CA); Martin Paul Onrot, Boynton Beach, FL (US); Antonio Vitale, LaSalle (CA); Teresa Champlin, Concordia, KS (US); Melva Nondorf, Belleville, KS (US); Melissa Novak, Narka, KS (US)

(73) Assignee: Scott Specialties, Inc., Belleville, KS (US)

(21) Appl. No.: 15/621,594

(22) Filed: Jun. 13, 2017

Related U.S. Application Data
(60) Provisional application No. 62/349,182, filed on Jun. 13, 2016.

(51) Int. Cl. A61F 5/01; A61F 5/058; A61F 5/00

(52) U.S. Cl. CPC A61F 5/0118; A61F 5/05866; A61F 5/01; A61H 2201/169; A61F 5/00

(57) ABSTRACT

A moldable wrist brace. The brace includes a pocket with a moldable insert disposed therein. The pocket and insert extend substantially the full length of the brace to maximize the length of the user's wrist supported thereby. A plurality of straps and buckles are provided for retaining the brace on the user's wrist and providing a desired compression thereon. The straps include locking tabs that once inserted through the buckles resist withdrawal of the straps therefrom. One-handed donning of the brace is thus simplified by retention of the straps in engagement with the buckles.

### CROSS-REFERENCE TO RELATED APPLICATIONS

This application claims the benefit of U.S. Provisional Patent Application No. 62/349,182 filed Jun. 13, 2016, the disclosure of which is hereby incorporated herein in its entirety by reference.

### BACKGROUND

A common practice for treatment of joints, bones, muscles, and the like includes immobilizing or supporting the area to be treated. A variety of devices are available for such treatments. The devices can be configured to completely immobilize the joint or to simply support the body part to relieve stresses thereon.

Removable devices for temporary or intermittent support are useful as treatment during activities that cause or exacerbate a particular ailment. The devices can be worn during the activity and then removed upon completion thereof. Such available devices can be problematic because they may not fit well, do not provide a sufficient support or compression, and are difficult to put on. Generally such devices are configured based on an average or common size and shape of the respective body part based on a large population of patients. The devices thus do not necessarily provide a perfect fit for all patients. And the devices often employ a variety of straps or ties that must be stretched and coupled to attach the device around the body part. The straps can be difficult to couple while also holding the device in position, especially when being applied to an arm or wrist and thus requiring the stretching and coupling to be completed one-handed while also holding the brace in the desired position.

### SUMMARY

Exemplary embodiments are defined by the claims below, not this summary. A high-level overview of various aspects thereof is provided here to introduce a selection of concepts that are further described in the Detailed-Description section below. This summary is not intended to identify key features or essential features of the claimed subject matter, nor is it intended to be used in isolation to determine the scope of the claimed subject matter. In brief, this disclosure describes, among other things, a wrist brace that includes a moldable insert and a plurality of fastening straps with locking tabs.

The wrist brace includes a body panel that is sized and shaped to wrap around the wrist of a user. The body panel includes a pocket in which a moldable insert is disposed. The insert is formed from a material that is substantially rigid or resilient at ambient temperatures, but, when heated, can be molded or shaped to a desired form, e.g. molded to the form of the user's wrist. After molding, the insert is installed into the pocket to provide the wrist brace with a custom-fit to the user.

The fastening straps are each comprised of mating sections of hook-and-loop fastener strips, i.e. a loop section and a hook section. One end of the loop section is coupled to the body panel and the opposite end is coupled to the hook section at a location spaced apart from but adjacent to a first end of the hook section. The first end of the hook section is thus a free end that overlies the loop section and forms locking tab.

In use the second end of the hook section is inserted through a buckle on the body panel. The strap is pulled through the buckle a sufficient distance to pull the locking tab through the buckle. The locking tab is deflected to lie on top of the loop section by the buckle to allow passage through the buckle. Once pulled through the buckle, the locking tab extends at least partially away from the loop section of the strap and the free end thereof is directed toward the buckle. When the strap is moved toward withdrawal from the buckle the locking tab engages and is deflected away from the loop section by the buckle and obstructs withdrawal of the strap through the buckle.

### DESCRIPTION OF THE DRAWINGS

Illustrative embodiments are described in detail below with reference to the attached drawing figures, and wherein:

FIG. 1 is an exterior-side plan view of a moldable wrist brace depicted in accordance with an exemplary embodiment;

FIG. 2 is a plan view of a moldable insert configured for use in the moldable wrist brace of FIG. 1 and depicted in accordance with an exemplary embodiment;

FIG. 3 is an interior-side plan view of the moldable wrist brace of FIG. 1 with a moldable insert inserted therein in accordance with an exemplary embodiment;

FIG. 4 is a perspective view of the moldable wrist brace of FIG. 1 applied to the wrist of a user in accordance with an exemplary embodiment; and

FIG. 5 is perspective view depicting the moldable wrist brace of FIG. 1 applied to the wrist of a user with the straps in an uncoupled state and with the locking tabs engaging buckles to resist withdrawal of the straps from the buckles in accordance with an exemplary embodiment.

### DETAILED DESCRIPTION

The subject matter of select exemplary embodiments is described with specificity herein to meet statutory requirements. But the description itself is not intended to necessarily limit the scope of claims. Rather, the claimed subject matter might be embodied in other ways to include different components, steps, or combinations thereof similar to the ones described in this document, in conjunction with other present or future technologies. Terms should not be interpreted as implying any particular order among or between various steps herein disclosed unless and except when the order of individual steps is explicitly described. The terms “about” or “approximately” as used herein denote deviations from the exact value by +/−10%, preferably by +/−5% and/or deviations in the form of changes that are insignificant to the function.

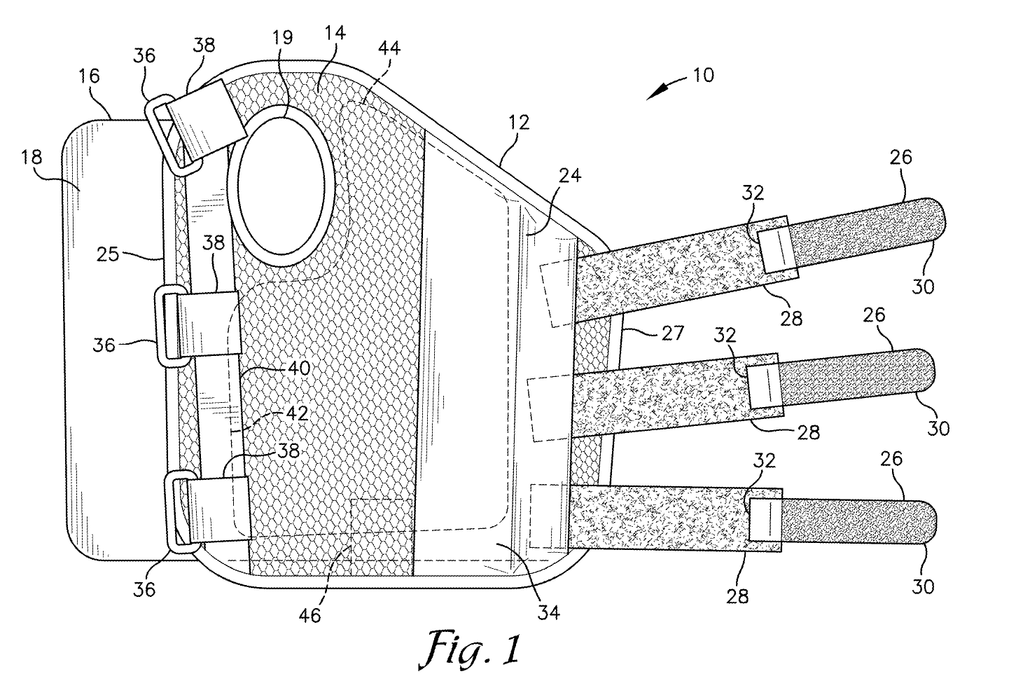

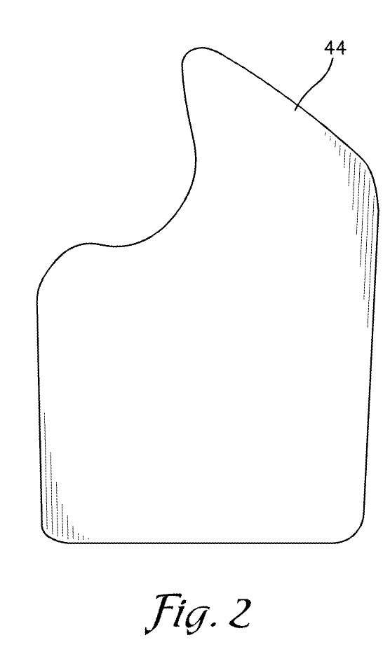

With reference to FIGS. 1-5, a moldable wrist brace 10 is described in accordance with an exemplary embodiment. The brace 10 is described herein with respect to applications in which the brace 10 is configured for use on the wrist of a user for at least partially supporting the hand and/or wrist. However it is understood that the brace 10 may be configured for application to a variety of other body parts, such as the ankle, without departing from the scope of embodiments described herein.

The brace 10 includes a body panel 12 formed from a plurality of material swatches including an exterior body swatch 14 and an interior body swatch 16. The exterior and interior body swatches 14, 16 have substantially the same overall outline or form, but for a flap 18 included on the interior body swatch, and are configured to wrap around the wrist of a user. Both the exterior body swatch 14 and the interior body swatch 16 form a thumbhole 19 extending therethrough. The thumbhole 19 is sized large enough to enable full dexterity of the user's thumb and to avoid application of pressure on the carpometacarpal joint of the user.

The exterior body swatch 14 is layered on the interior body swatch 16 and is coupled thereto, such as by sewing along three edges of the exterior body swatch 14. One edge (the bottom edge as shown in FIG. 1) of the exterior body swatch 14 is left uncoupled to the interior body swatch 16 to form an opening 20 into a pocket 22 formed between the swatches 14, 16. One or more additional stitch lines 24 may be provided to further define the size and/or shape of the pocket 22, further couple the swatches 14, 16, and/or to act as additional stiffeners for the body panel 12.

The exterior and interior body swatches 14, 16 are preferably comprised of materials such as a neoprene, a nylon, and/or a spandex material and may incorporate three-dimensional knitted fabrics. The materials preferably include antibacterial and or anti-odor properties and are hypoallergenic. The materials may be selected to enhance comfort, breathability, insulation, compression, durability, and recovery among other characteristics.

The flap 18 extends beyond a first side edge 25 of the exterior body swatch 14. The flap 18 is sized to substantially span a gap between the opposing side edges of the exterior body swatch 14 when the brace 10 is wrapped around the wrist of a user. Although, the flap 18 is described herein as comprising a portion of the interior body swatch 16, the flap 18 might alternatively be formed by the exterior body swatch 14, both the exterior and interior body swatches 14, 16, or a separate swatch coupled to the body panel 12.

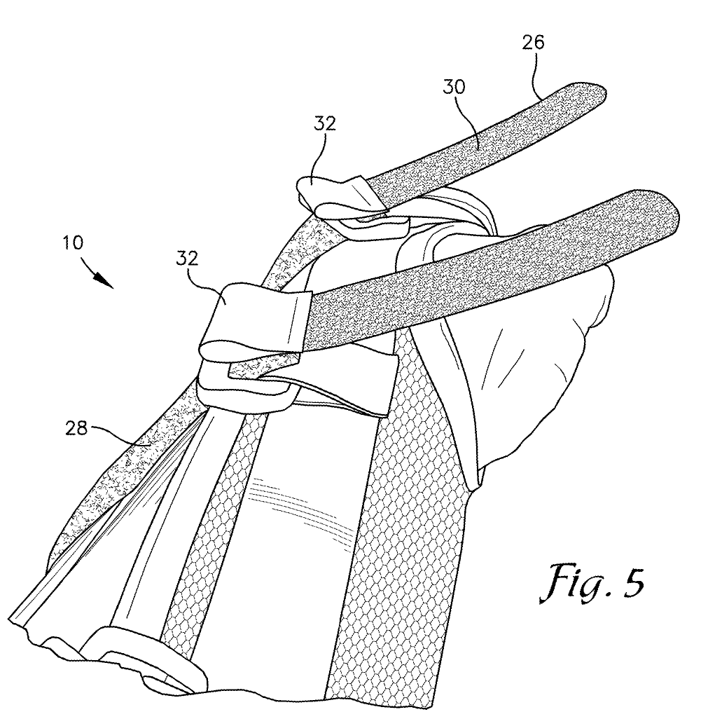

A plurality of straps 26 are coupled to the body panel 12 near a second side edge 27, opposite the flap 18. The straps 26 can be configured to employ an available coupling technique, including buckles, snaps, ties, or the like, but preferably comprise hook-and-loop fasteners. The straps 26 are also preferably formed from generally non-extensible materials, but are not necessarily so limited. As depicted in FIGS. 1-5, the straps 26 are each comprised of two sections, a loop section 28 and a hook section 30, each formed with the respective component of a hook-and-loop fastener. The loop section 28 is coupled at a first end to the exterior body swatch 14 near the second side edge 27. A second end of the loop section 28 is coupled to the hook section 30 at a location spaced apart from, but near a first end of the hook section 30. The first end of the hook section 30 thus overlaps at least a portion of the loop section 28. The first end is folded back onto itself to form a loop or locking tab 32 that overlies the loop section 28 of the strap 26, as depicted in FIG. 5. In another embodiment, the first end is not folded back onto itself and the locking tab 32 is thus formed by the free first end of the hook section 30. The locking tab 32 deflects at least partially away from the plane of the loop section 28.

A coupling swatch 34 is applied over a portion of the exterior body swatch 14 and overlaps the first ends of the straps 26. The coupling swatch 34 may be configured for engagement by the hook section 30 of the straps 26.

A plurality of buckles 36 are coupled along and/or adjacent the first edge 25 of the exterior body swatch 14 adjacent the flap 18. The buckles 36 are positioned for engagement by respective ones of the straps 26 when the brace 10 is wrapped around the wrist of the user. The buckles 36 comprise loops or rings, such as plastic or metal generally rectangular rings, coupled to the body panel 12 by a securement 38 comprising a short section of non-extensible material that is folded onto itself to pass through the buckle 36; ends of the securement 38 are attached to the exterior body swatch 14. It is understood that the buckles 36 may take a variety of forms depending on the form of the straps 26 and the coupling mechanism provided thereby. All such forms are within the scope of exemplary embodiments described herein.

A reinforcing swatch 40 is disposed on the exterior body swatch 14 near the attachment of the securement 38 to the exterior body swatch 14. The reinforcing swatch 40 reinforces the exterior body swatch 14 against forces applied thereto by the buckles 36 and securements 38 when tension is placed thereon. A stay 42 is also installed between the reinforcing swatch 40 and the exterior body swatch 14 to further reinforce the body 10.

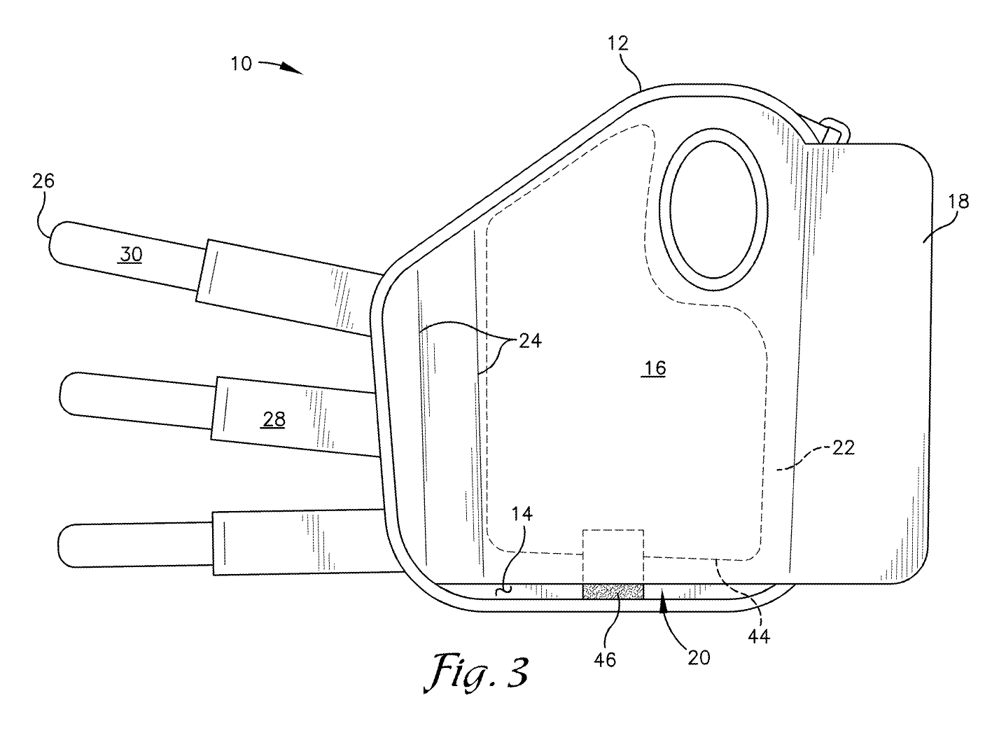

A moldable insert 44 is removably installed in the pocket 22. The insert 44 is comprised of a material, such as a polyvinyl chloride (PVC) material or similar plastic, composite or other material, that is substantially resilient and/or rigid at ambient or room temperatures. However, when heated, the material can be formed into a desired shape and then cooled to regain the original resilience or rigidity. The insert 44 is shaped to generally follow the perimeter of the pocket 22 so as to be insertable therein. The insert 44 and the pocket 22 are configured to extend substantially the full length of the brace 10 (extending generally parallel to and along the first and second edges 25, 27) to maximize the length along the wrist of the user that is supported thereby. The insert 44 and pocket 22 are also configured to wrap around about two-thirds of the circumference of the wrist of the user, however the insert 44 and pocket 22 may extend a further or lesser extent around the wrist. A fastener 46, such as a hook-and-loop faster may be provided along the opening 20 of the pocket 22 to retain the insert within the pocket 22, as depicted in FIGS. 1 and 3.

With continued reference to FIGS. 1-5, use of the moldable wrist brace 10 is described in accordance with an exemplary embodiment. The brace 10 may be provide in a variety of sizes to accommodate users of all sizes and/or body parts of all sizes. An appropriately sized brace 10 is selected for the user. If installed in the pocket 22, the insert 44 is removed from the pocket 22 and heated using, for example, an oven, a heat tray, a heat gun, or hot water to a temperature at which the insert material becomes at least partially pliable. For example, if the insert 44 is comprised of a PVC material, the material may become pliable at temperatures above about 71° C. Once the insert 44 has been sufficiently heated it is removed from the heat source and may be allowed to cool for a short time period, e.g. about 15 seconds.

The user's wrist or other body part on which the brace 10 is to be worn may be covered with a stockinette or other thin, cloth-like material to provide at least a partial barrier against the heat of the insert 44. The material should not be overly thick so as to avoid interfering with molding of the insert 44 to the form of the user's wrist. The heated insert 44 is placed on the user's wrist and gently molded by hand to follow the form of the wrist. The insert 44 is maintained on the wrist until it is no longer substantially pliable. The insert 44 can then be removed and allowed to cool. If desired the insert 44 can be reheated and remolded to the user's wrist. In another embodiment, the material comprising the insert 44 may not be remoldable.

The molded insert 44 is installed into the pocket 22 and the fastener 46 fastened to retain the insert 44 therein. The brace 10 is applied to the user's wrist by inserting the thumb of the user through the thumbhole in the body panel 12. The body 10 is wrapped around the user's wrist with the flap 18 positioned against the wrist and overlapped by the second edge 27 of the body panel 12. In one embodiment, the heated insert 44 is installed in the pocket 22 and the body 10 is applied to the user's wrist to mold the insert 44 to the user's wrist.

The distal ends of the straps 26 are inserted through the buckles 36 a distance sufficient to draw the locking tabs 32 thereon through the buckles 36. As the locking tab 32 moves through the buckle 36 the locking tab 32 is pressed toward the loop section 28 of the strap 26 and lies generally parallel to the loop section 28. Upon passing through the buckle 36 the locking tab 32 deflects at least partially away from the loop section 28 with the end thereof (e.g. the loop formed by the first end of the hooked section 30) being directed toward the buckle 36.

When the strap 26 is moved to withdraw from the buckle 36 the locking tab 32 contacts and is deflected away from the loop section 28 by the buckle 36. Interaction between the locking tab 32 and the buckle 36 thus resists withdrawal of the strap 26 from the buckle 36. As such, a user is aided in donning the brace 10, especially when doing so one-handed, because the straps 26 are retained in engagement with the buckles 36. The user can thus slide on the brace 10 by inserting the hand into the pre-wrapped brace 10 rather than being required to wrap the brace 10 around the wrist.

To remove the straps 26 from the buckles 36, the locking tab 32 can be held against the loop section 28 and inserted through the buckle 36. Or additional withdrawal force may be applied to the strap 26 to cause the locking tab 32 to be folded onto the hook section 30 and forced through the buckle 36. This method may require additional force because four layers of the strap 26 (e.g. three layers of the hook section 30 and one layer of the loop section 28) must be forced through the opening in the buckle 36.

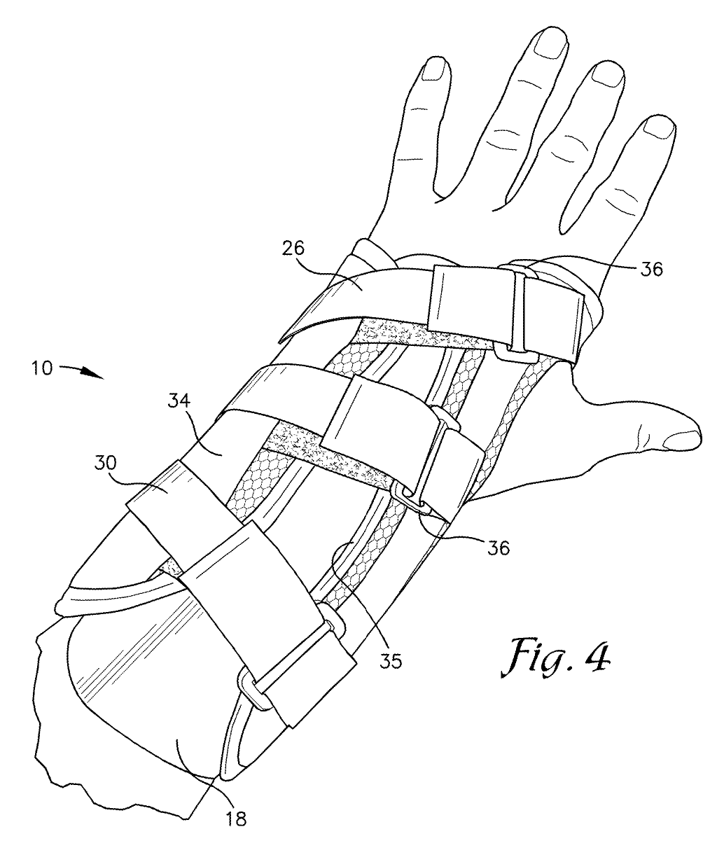

As depicted in FIG. 4, after passing through the buckles 36, the straps 26 are folded back onto themselves to engage the hook section 30 with the loop section 28 and complete securement of the brace 10 on the wrist. The straps 26 may be pulled as tightly as desired by the user to provide the desired level of compression and support to the wrist. The distal end of one or more of the straps 26 may extend beyond the first end of the respective loop section 28; the additional length of the hook section 30 may engage the coupling swatch 34 and couple thereto. Such may avoid the distal ends of the straps 26 from extending away from the exterior of the brace 10 and being bothersome by catching on clothing or other items.

Many different arrangements of the various components depicted, as well as components not shown, are possible without departing from the scope of the claims below. Embodiments of the technology have been described with the intent to be illustrative rather than restrictive. Alternative embodiments will become apparent to readers of this disclosure after and because of reading it. Alternative means of implementing the aforementioned can be completed without departing from the scope of the claims below. Identification of structures as being configured to perform a particular function in this disclosure and in the claims below is intended to be inclusive of structures and arrangements or designs thereof that are within the scope of this disclosure and readily identifiable by one of skill in the art and that can perform the particular function in a similar way. Certain features and sub-combinations are of utility and may be employed without reference to other features and sub-combinations and are contemplated within the scope of the claims.

What is claimed is:

1. A joint immobilizing brace comprising:
a panel configured to at least partially envelop a portion of a living body that includes a joint;
a pocket formed by the panel, the pocket extending across the joint and at least partially surrounding the portion of the living body; and
an insert disposed in the pocket and substantially filling the pocket, at an elevated temperature the insert being moldable to substantially conform to the portion of the living body and at room temperature being substantially rigid.

2. The joint immobilizing brace of claim 1, further comprising:
a plurality of straps, each of the straps including a first portion and a second portion, a first end of the first portion being coupled to the panel, a second end of the first portion being coupled to the second portion at a location that is spaced apart from a first end of the second portion, the first end of the second portion overlapping the first portion of the strap and forming a locking tab, a second end of the second portion being a free end.

3. The joint immobilizing brace of claim 2, wherein the first portion of each of the straps comprises one of a hook-portion or a loop-portion of a hook-and-loop fastener and the second portion comprises the other of the hook-portion or the loop-portion.

4. The joint immobilizing brace of claim 2, wherein the first end of the second portion is doubled over onto itself to form a loop that overlaps the first portion of the strap.

5. The joint immobilizing brace of claim 2, further comprising:
a buckle coupled to the panel and aligned with a respective one of the plurality of straps, the buckle being configured to receive the strap therethrough, the locking tab formed by the first end of the second portion lying against the first portion and enabling passage of the locking tab through the buckle in an insertion direction, in an opposite removal direction the locking tab being pivoted away from the first portion and obstructing passage of the strap through the buckle in the removal direction.

6. The joint immobilizing brace of claim 1, further comprising:
a flap extending from a first edge of the panel, the panel being wrapped around the portion of the living body and a second opposite edge of the panel overlapping at least a portion of the flap.

7. The joint immobilizing brace of claim 1, further comprising:
a securement swatch disposed on an outer surface of the panel, the securement swatch being engageable by the hook-portion of the strap.

8. The joint immobilizing brace of claim 1, wherein the panel is comprised of an interior swatch and an exterior swatch that are secured together generally along three edges thereof to form the pocket, the insert being insertable into the pocket and between the interior and exterior swatches along a fourth edge of the panel.

9. The joint immobilizing brace of claim 8, wherein one or both of the interior and exterior swatches are comprised of a material that imparts antimicrobial characteristics.

10. The joint immobilizing brace of claim 1, wherein the portion of the living body comprises a human wrist.

11. The joint immobilizing brace of claim 1, wherein the pocket and the insert extend substantially the full length of the brace to maximize support to the joint.

12. The joint immobilizing brace of claim 5, wherein at least three sets of straps and buckles are included to provide sufficient compression to the joint.

13. A joint immobilizing brace comprising:
a panel configured to at least partially envelop a portion of a living body that includes a joint;
a pocket formed by the panel, the pocket extending across the joint and at least partially surrounding the portion of the living body;
an insert disposed in the pocket and substantially filling the pocket, at an elevated temperature the insert being moldable to substantially conform to the portion of the living body and at room temperature being substantially rigid; and
a plurality of straps, each of the straps including a first portion and a second portion, a first end of the first portion being coupled to the panel, a second end of the first portion being coupled to the second portion at a location that is spaced apart from a first end of the second portion, the first end of the second portion overlapping the first portion of the strap and forming a locking tab, a second end of the second portion being a free end.

14. The joint immobilizing brace of claim 13, wherein the insert is comprised of a sheet of polyvinyl chloride material.

15. The joint immobilizing brace of claim 13, wherein the first portion of each of the straps comprises one of a hook-portion or a loop-portion of a hook-and-loop fastener and the second portion comprises the other of the hook-portion or the loop-portion.

16. The joint immobilizing brace of claim 13, wherein the first end of the second portion is doubled over onto itself to form a loop that overlaps the first portion of the strap.

17. The joint immobilizing brace of claim 13, further comprising:
a buckle coupled to the panel and aligned with a respective one of the plurality of straps, the buckle being configured to receive the strap therethrough, the locking tab formed by the first end of the second portion lying against the first portion and enabling passage of the locking tab through the buckle in an insertion direction, in an opposite removal direction the locking tab being pivoted away from the first portion and obstructing passage of the strap through the buckle in the removal direction.

18. The joint immobilizing brace of claim 13, wherein at least three straps are included to provide substantial compression to the joint.

19. The joint immobilizing brace of claim 11 wherein the pocket and insert extend substantially the full length of the brace to maximize support to the joint.

20. A joint immobilizing brace comprising:
a panel configured to at least partially envelop a wrist joint of a human body;
a pocket formed by the panel, the pocket extending across the wrist joint and at least partially surrounding the wrist; and
an insert disposed in the pocket and substantially filling the pocket, at an elevated temperature the insert being moldable to substantially conform to the wrist and at room temperature being substantially rigid to resist flexure of the wrist joint, and extending substantially the full length of the brace to maximize support to the wrist.

21. The joint immobilizing brace of claim 20, further comprising:
a plurality of straps, each of the straps including a first portion that comprises a loop-portion of a hook-and-loop fastener and including a second portion that comprises a hook-portion of the hook-and-loop fastener, a first end of the first portion being coupled to the panel, a second end of the first portion being coupled to the second portion at a location that is spaced apart from a first end of the second portion, the first end of the second portion overlapping the first portion of the strap and forming a locking tab, a second end of the second portion being a free end; and
a buckle coupled to the panel and aligned with a respective one of the plurality of straps, the buckle being configured to receive the strap therethrough, the locking tab formed by the first end of the second portion lying against the first portion and enabling passage of the locking tab through the buckle in an insertion direction, in an opposite removal direction the locking tab being pivoted away from the first portion and obstructing passage of the strap through the buckle in the removal direction.

22. The joint-immobilizing brace of claim 20, wherein said panel includes a thumb hole sized to allow full dexterity of a thumb and avoid pressure to a carpometacarpal joint.

23. The joint-immobilizing brace of claim 21 wherein at least three straps and buckles are included to provide sufficient compression to the wrist.

24. The joint-immobilizing brace of claim 21, wherein engagement of the locking tab with the buckle to obstruct passage of the strap through the buckle in the removal direction retains engagement of the straps with the buckles during donning of the brace.

25. The joint-immobilizing brace of claim 24, wherein the brace is donned by a user using only one hand.
